(19) United States
(12) Patent Application Publication
(10) Pub. No.: US 2011/0106230 A1
(43) Pub. Date: May 5, 2011

(54) PLACEMENT DEVICE FOR INSERTING MEDICAL IMPLANTS SUCH AS ELECTRODE LINES

(76) Inventor: Erhard Flach, Berlin (DE)

(21) Appl. No.: 12/611,944

(22) Filed: Nov. 4, 2009

(51) Int. Cl. A61N 1/00; A61B 17/00

(52) U.S. Cl. 607/116; 607/149

(57) ABSTRACT

A placement device for precise placement of a medical implant into a living organism includes an elongated body with a distal end and a proximal end. The placement device can be introduced into the living organism through an aditus, so that the proximal end remains outside of the living organism. The distal end has an anchor which can be firmly anchored in the body tissue in removable manner. The medical implant includes an elongated hollow housing with a distal and a proximal end, whereby the hollow housing can translatably receive the placement device therein. The opening at the distal end is provided with a sealing unit for sealing the elongated hollow housing with respect to the placement device.

### FIELD OF THE INVENTION

The invention concerns a placement device for precise insertion (permanent or temporary) of medical implants, such as electrode lines, into a living organism. A preferred version of the placement device has an elongated device body including a distal device end and a proximal device end, whereby the placement device is configured in such a way that it can be inserted into the living organism through an aditus (i.e., an opening in the body), so that the proximal device end remains outside of the living organism.

Further, the invention concerns a medical implant (e.g., an electrode line) for permanent or temporary disposition in a living organism wherein the implant includes a hollow implant housing with a distal implant end and a proximal implant end, and with an implant passage is extending along the longitudinal axis of the hollow housing. The implant housing is configured in such a way that it can receive and guide the placement device, and it includes openings at its respective proximal and distal implant ends through which the placement device can pass.

### BACKGROUND OF THE INVENTION

Placement devices are used to bring electrode lines to a desired implantation site into a living organism, where they are then secured. To illustrate, a so-called guide wire is sometimes extended through the venous system into a heart chamber or a coronary blood vessel, with an inserted electrode line then extending alongside the guide wire to be secured in the body tissue (such as in the coronary wall). Problems arise when certain specific areas are targeted to receive the electrode line for subsequent stimulation, for example, when a physician would like to affix an electrode line high on the septum in the proximity of the His Bundle. Generally, the electrode line must be brought to the fixation site with a guiding sheath such as a catheter. Because of the narrow passageways involved, the bending radii within the guiding sheath are small, and it is therefore difficult to guide the electrode line toward the target area owing to increasing friction between the electrode line and the guiding sheath (which prevents the electrode line from sliding forward). The physician also experiences difficulties in urging the tip of the electrode line against the cardiac wall, since the tip is (preferably) at or near a 90° orientation relative to the axis of travel of the electrode line, and the sheath is not sufficiently rigid that one can swing it to urge the electrode line tip into the cardiac wall with any significant force. Additionally, a catheter or other sheath—even one with a high bending radius—is usually only able to guide electrodes toward the targeted area at a tangential or acute angle. It is then very difficult (or often impossible) to affix standard electrodes, such as active fixable screw electrodes, because the screw must be oriented near a perpendicular to the targeted area to generate sufficient contact pressure for engagement.

Likewise, similar methods can be used to fasten an electrode line at the outer wall of the heart (epicardium). For this purpose, a guiding sheath is inserted through the chest wall, and the electrode line is guided therein and attached to the epicardium. In order to get to the heart wall is from the outside of the body, the sheath and electrode line must pass through the relatively narrow space between the chest wall and ribs. As a result, the electrode line does not approach the epicardium at a right angle, and an attempt must be made to fasten the electrode to the heart wall as the electrode approaches tangentially to the heart wall. Again, a conventional screw-in electrode line cannot be fastened at such an angle, since the screw must be near perpendicular to successfully achieve penetration.

### SUMMARY OF THE INVENTION

The present invention therefore seeks to provide a placement device, and an electrode line or other medical implant that works with the placement device, that promotes safe and precise anchoring of the medical implant in the body tissue in such a way that the user can more readily attain fastening of the electrode line after insertion.

This objective is furthered by a placement device for precise (and permanent or temporary) insertion of a medical implant into a living organism, which includes an elongated device body with a distal device end and a proximal device end. The placement device is configured in such a way that it can be inserted into the living organism through an aditus (opening in the living body) so that the proximal device end remains outside of the living organism, and the distal device end has an anchor with which the placement device can be firmly connected in a removable manner to body tissue. Preferably, the distal anchor is designed to penetrate into the body tissue, i.e. it is preferably pointed, and most preferably has the shape of a needle. The distal device end of the placement device can be temporarily affixed to the myocardium as a result of penetration by the anchor.

Once the placement device is affixed in this way, an electrode line or similar medical implant can then be secured in the tissue. The electrode line/implant includes a hollow implant housing extending along a longitudinal axis between a distal implant end and a proximal implant end, with openings at the implant ends through which the placement device can pass. The implant housing is configured to receive the placement device and removably sheath it therein. The opening at the distal implant end has a sealing unit for sealing the implant housing, which can be reversibly penetrated by the anchor of the placement device.

In another preferred version of the invention, the anchor has a bend that extends in the lateral direction, with the bend preferably having a radius of 5 mm to 20 mm and/or a circular arc with an angle measuring between 70° to 90°. By means of the bend, the electrode line or other implant can be brought to the implantation site via the placement device by guiding it (for example) in the narrow space between chest passage and pericardium, then in the particularly narrow space in the pericardium to tangentially approach the epicardium, to subsequently be fastened at an appropriate angle onto the myocardium. The placement device allows the necessary penetration forces to be applied to the implant.

To make production easier and to better allow use of conventional implants, the device body and the anchor of the placement device are preferably flexible, preferably consisting of a wire, with a wire having a diameter smaller than 0.36 mm being particularly preferred.

To better avoid injury during insertion of the placement device, the placement device preferably has a locking mechanism at the proximal end, which allows direct or indirect removable locking with the medical implant. This allows selection of the relative position between the placement device and the medical implant and/or prevention of displacement between the placement device and implant.

In another version of the invention, the placement device further includes a guiding sheath which translatably carries the placement device therein, with the sheath having an elongated hollow sheath housing with a distal sheath end and a proximal sheath end, and a sheath passage extending continuously therebetween along a longitudinal axis. The sheath housing is configured so that it can receive the placement device and guide it reversibly along its length, and/or retain the placement device. Further, the sheath includes openings at the proximal and distal sheath ends, which are aligned with the device passage of the device housing and through which the placement device can pass. The sheath further includes a locking unit at the proximal sheath end which directly or indirectly allows selective locking with the placement device, in order to fix the relative position between the placement device and the sheath, and/or to prevent displacement between the placement device and the sheath. Preferably, the sheath is designed as sleeve, such as an elastic, bendable Mandrin sleeve. Owing to the sheath, the insertion of an electrode line or similar implant can be further eased since the placement device is then supported by the sheath and significantly more stable. Further, as a result of the sheath, the insertion and placement of the electrode line can be simplified significantly.

The invention also involves a medical implant for disposition in a living organism, which includes a hollow implant housing with openings at distal and proximal implant ends, and an implant passage extending continuously between the openings along a longitudinal axis. The implant passage is configured in such a way that it can receive a placement device for precise permanent or temporary insertion of medical implants in a living organism (with the placement device including an elongated device body with distal and proximal device ends, and an anchor at the distal device end allowing the placement device to be anchored in body tissue). The placement device can enter the implant passage from one of the openings (e.g., at the proximal implant end), and the distal implant end has a sealing unit that can be reversibly penetrated by the anchor of the placement device, and which seals the implant housing about the penetrating anchor.

The electrode line/implant preferably has a stop arrangement within its implant passage, situated adjacent and anterior to the distal sealing unit. The stop preferably has a thrust bearing opening onto a narrower bore, with the stop being configured such that the anchor of the placement device can pass through the bore, with any sheath situated over the placement device being halted by/at the thrust bearing. The sheath can therefore be inserted into the electrode line/implant with the sheath stopping at the thrust bearing upon an exertion of force extending along the sheath's length in the distal direction. Upon further exertion of force on the sheath, the implant, which preferably consists of pliable or thin material throughout, is driven in the distal direction by the sheath until it reaches the implantation site at which it can be affixed.

Preferably, the medical implant is a medical electrode line which includes at least one electrical pole for emitting electrical pulses onto the body tissue of the living organism at the distal implant end; one or more conductors, each being electrically connected with one of the poles and extending from the respective pole to the proximal implant end; and a connection unit at the proximal end which is connected with each of the electrical conductors in order to establish an electrical connection with an implantable or external electrical placement device. Electrode lines of this nature are currently used in modern medicine in a variety of forms. For example, they can be used for electrical stimulation in or at a heart in order to ensure the safe function of the heart. Further, such electrode lines can also be used in the nervous system or in a brain. These lines must be extremely fine, and thus their ability to be smoothly guided along a Mandrin sleeve with minimal stress is highly important.

Preferably, the electrode line/implant is a permanently implantable screw electrode with a fixed or retractable screw fixation at the distal end, whereby the screw fixation preferably forms one of the electrical poles. Most preferably the screw fixation is sheathed in a screw sheath located at the distal implant end, and which at least partially radially surrounds the distal implant opening. As a result, the electrode line becomes easily implantable even under difficult spatial conditions.

In another version of the invention, a guiding sheath similar to the one described above includes a primary passage for guiding the placement device, and also includes a secondary passage which also extends longitudinally within the sheath housing adjacent the primary passage, and between the distal and proximal sheath ends. The secondary passage is configured to receive and carry a medical device (e.g., an electrode line or other medical implant). The sheath may include a locking unit at the proximal sheath end which selectively allows locking (directly or indirectly) with the placement device, thereby allowing selection of the relative positioning between the placement device and the sheath, and/or preventing displacement between the placement device and the sheath. The secondary passage may therefore be used to precisely place an electrode line, while a placement device in the primary is passage holds the distal end of the sheath at a desired location alongside a tissue target.

Targeted insertion of other medical devices via the secondary passage is possible, for example, medication delivery or other sheaths, sensors, tissue removal/biopsy devices, dilators, and the like.

In order to more easily use the guiding sheath for the implantation of electrode lines, which have a relatively large connection unit for connecting to an implantable or external medical device, the hollow housing is preferably formed of material which is easily peeled or slit, so that the wall of the passage(s) can be opened. As a result it is possible, after successfully securing an implant, to open the passage(s) completely along the length of the sheath so that (for example) the sheath can be completely removed after the release of the anchor, with the implant remaining in the body.

Additional goals, features, advantages and possible applications of the invention are given by the following description of exemplary versions of the invention in conjunction with the figures. The invention encompasses all described and/or pictorially illustrated features by themselves or in any combination, regardless of their characterization in the foregoing and following discussions.

### BRIEF DESCRIPTION OF THE DRAWINGS

The drawings depict

FIG. 1a, FIG. 1b: Simplified schematic illustrations of temporary fixation with an exemplary placement device;

FIG. 2: The distal end of an exemplary placement device with a fixable electrode line;

FIG. 3a, FIG. 3b: A second exemplary version of the placement device with a fixable electrode line and a Mandrin sleeve;

FIG. 4: The distal end of a third exemplary version of the placement device with a guiding sheath.

### DETAILED DESCRIPTION OF PREFERRED VERSIONS OF THE INVENTION

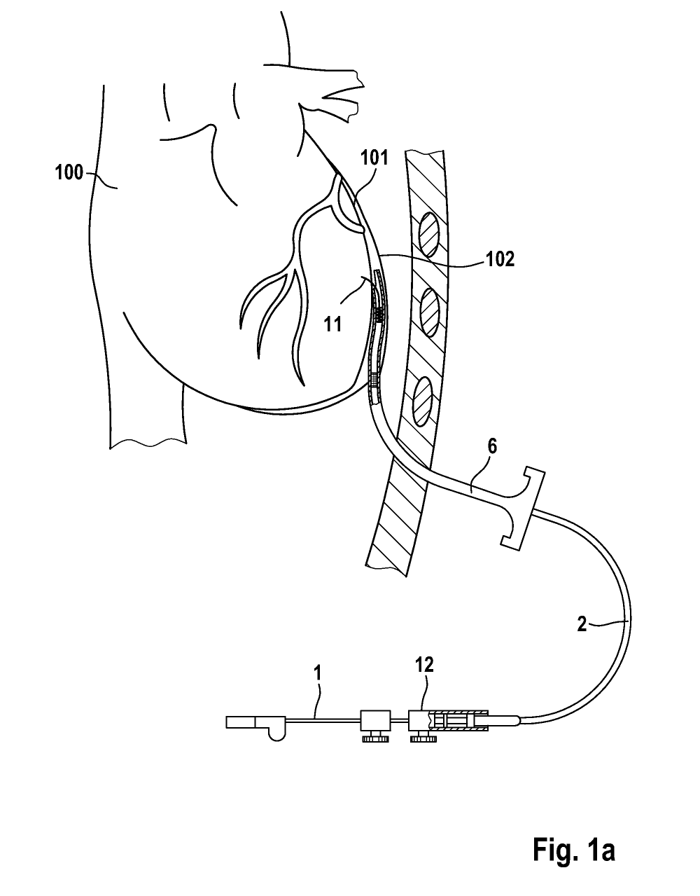

FIGS. 1a and 1b illustrate an exemplary use of an exemplary placement device 1 for precise placement of a medical implant (screw electrode line) 2 on the epicardium 101 of a heart 100. With the help of an elongated sheath 6 (FIG. 1b), the chest passage is opened and the distal section 61 of the sheath 6 is pushed forwardly up to the pericardium 102, which is then penetrated by sheath 6, so that the epicardium 101 is reached. At the distal end 61, the sheath 6 has an opening in the lateral direction which comes to directly abut the tissue of the epicardium 101.

In FIG. 1a, the problem that is to be addressed with the placement device 1 becomes clear: in the pericardium 102, it is generally only possible to reach the tissue of the epicardium 101 tangentially, without risking (for example) an unintentional puncture of the heart and resultant life-threatening conditions. It is exceedingly difficult to anchor a screw electrode line 2 using this tangential approach, as the screw fixation does not have any reasonable possibility of penetrating the tissue of the epicardium 101.

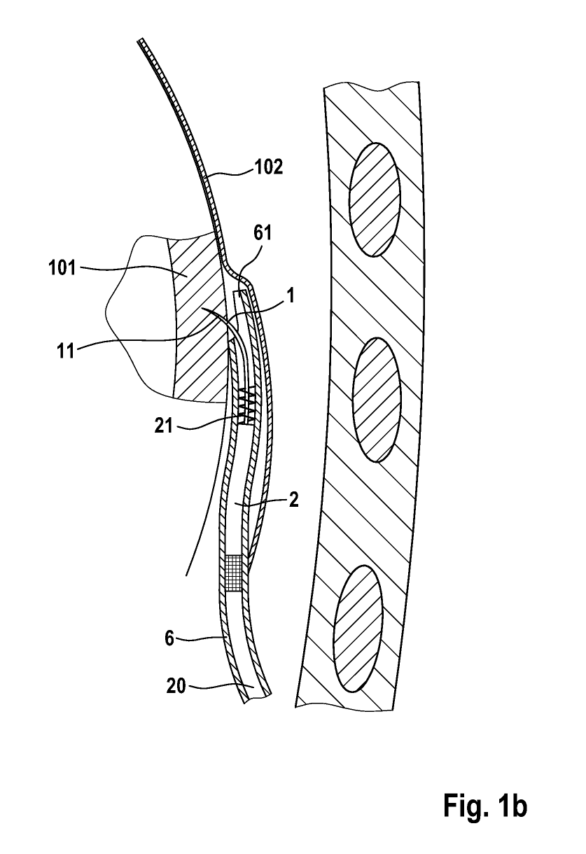

Referring back to FIG. 1b and to FIG. 2, after the sheath 6 has been placed at the desired location, a placement device 1 with an anchor 11 is slid through a passage 22 within the electrode interior 20 of the electrode line 2. The placement device 1 is then secured with respect to the electrode line 2 with the help of a locking unit (element 12 of FIG. 1a) such that the anchor 11 does not protrude laterally out of the passage 22 of the electrode line 2. In this condition, the fastened placement device 1 and the electrode line 2 are slid through the sheath 6 to its distal end 61. Once the distal end of the electrode line 2 has arrived at the distal end 61 of sheath 6, the electrode line 2 can be locked with suitable means with respect to the sheath 6, while the locking unit 12 securing the placement device 1 with respect to the electrode line 2 is released. The bent and pointed anchor 11 can then project out of the distal and lateral opening is of sheath 6 and penetrate the epicardium 101 to a desired depth to be anchored there. As a result of this anchoring and the pre-bending of the anchor 11, an arc is formed that penetrates at an angle of 70 to 90 degrees into the epicardium 101. The electrode line 2 can then be slid forward and can be fastened as the screw fixation 21 of electrode line 2 now has the possibility of engaging the epicardium at or near-perpendicular angle. After fastening the electrode line 2, the anchor 11 is pulled out of the epicardium 101 and the placement device 1 is removed together with the sheath 6.

FIG. 2 shows a permanently implantable electrode line 2 with fixed and retractable screw fixation 21 and a placement device 1 for precise permanent or temporary insertion of implants. In this and in the other depicted versions, placement device 1 is designed as wire and has a long extended body 10 with an anchor 11, configured as a needle-shaped and pre-bent elastic tip. The anchor 11 has a radius of a minimum of 5 mm up to a maximum of 20 mm and is bent into a circular arc at an angle of 70 to 90 degrees. The user can select/set the orientation of the arc as desired. The diameter of the wire 1 has a conventional size that is approximately 0.36 mm. A marking can be provided at placement device 1, with the aid of which it can be determined how far the anchor 11 was moved out.

The electrode line 2 has at least one pole that can also be formed by the screw fixation, and an electrode interior 20, including a continuous passage 22 with a distal and a proximal opening. The passage 22 is configured in such a way that placement device 1 can be translatably inserted therein. Further, electrode line 2 has a sealing unit 23 at the distal end that prevents blood from entering and which can only be penetrated reversibly by the anchor 11 of the placement device 1. Thus, the wire 1 can protrude at the distal end of the electrode line 2 and be temporarily anchored in the tissue of the living organism.

The fixation screw 21 is attached at the distal end of electrode line 2, to translate within a rigid housing 24, so that a combined rotation/translation of the fixation screw 21 accomplishes a screwing in or a screwing out of the body tissue. As the result of a rotational input at the proximal end of the electrode line 2, the fixation screw 21 can be screwed in or out. The housing 24 thereby experiences an elongation along the electrode line 2 that is smaller than 15 mm. At its proximal end, the electrode line may bear a plug connection for engagement with a connection unit (not shown here). With this connection unit, most often in the form of a standard plug according to IS-1, DF-1 and/or IS-4 standards, an electrical connection can be established with an implantable or external electrical device (heart pacemaker, cardioverter/defibrillator, brain or nerve pacemaker or other electrical device).

Looking to FIG. 1a, the placement device 1 can be removably connected to the proximal end of the electrode line 2 by means of a locking unit 12 that is located on it in order to prevent the anchor 11 from unintentionally exiting at the distal end of the electrode line 2. Such locking can take place by means of screwing, snapping or clamping. Further, at the proximal end of the electrode line 2 or in its proximity, a marking that is visible from the outside of the body can be provided with which it can be determined if the electrode line 2 has advanced through the sheath 6 up to the cardiac wall.

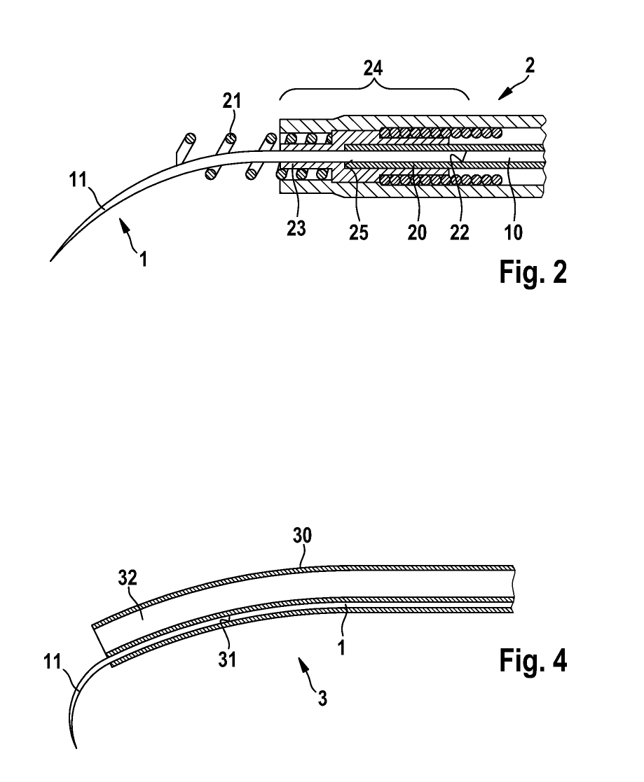

FIGS. 3a and 3b show another version of the placement device 1 for precise permanent or temporary insertion of implants. Placement device 1, provided in the form of a wire, runs displaceably in a sheath 4 provided in the form of a Mandrin guiding sleeve. Such a sheath 4 is used for guiding the electrode line 2 more easily to the implantation site, and has a hollow body 40 with a continuous passage 41 extending from a proximal end 43 to a distal end 42.

So that the electrode line 2 can be guided by sheath 4, electrode line 2 additionally has a stop 25 (FIG. 2) at the distal end directly proximal to the sealing unit 23, with the stop 25 including a bore and a thrust bearing. Such a stop is known, for example, from EP 1 356 845 A1 (US 20030229277 A1) or EP 1 452 200 A1. The bore is designed in such a way that the anchor 11 of the wire 1 can pass through, while the sheath 4 stops at the thrust bearing, whereby it becomes possible that the electrode line 2 can be guided with the help of sheath 4. The sheath 4 has an interior diameter that permits displaceable guiding of a wire 1 with a diameter of 0.36 mm, or more preferably an inner diameter that is a little larger than 0.36 mm, preferably 0.4 mm.

Further, the sheath 4 is provided with a locking unit 44 at its proximal end whereby the wire 1 can be removably connected with sheath 4 in order to prevent that the anchor 11 unintentionally exits at the distal end 42 of sheath 4.

The wire 1 can be removably connected to the electrode line 2 at its proximal end by means of a locking unit 12 provided on wire 1, in order to prevent a displacement of the temporary connection of wire 1 and sheath 4 relative to electrode line 2. Here too, the lockings 12 and 44 can take place by means of screwing, snapping or clamping. After the release of lock 44, the anchor 11 of wire 1 can pass through the stop 25 and the sealing 23 at a defined length, and can move out of the distal end of the electrode line and enter into the tissue of the living organism.

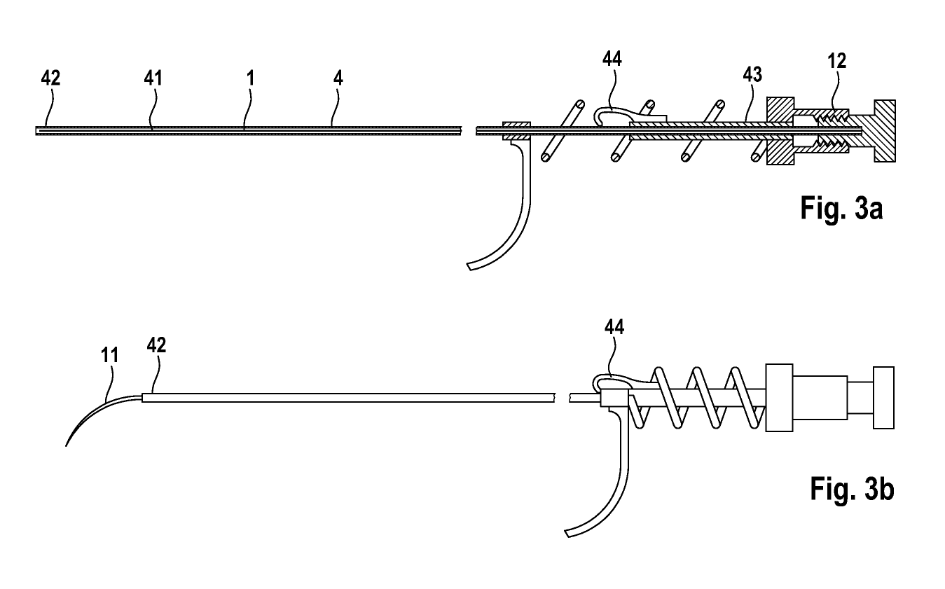

FIG. 4 shows a further version including a guiding sheath 3 and a placement device 1. The guiding sheath 3 has a double hollow housing 30 with two passages 31 and 32 that extend in parallel, whereby passage 31 is designed for guiding placement device 1 in a displaceable manner. The additional passage 32 is designed so that it has no connection with passage 31. Further, passage 32 is designed in such a way that it can, for example, guide an active fixable electrode line. Moreover, passage 32 is designed to be peeled or slit. Both passages are hollow throughout, with each having openings at the distal and proximal end of the sheath 3. At its proximal end, the guiding sheath 3 has a lock that catches the placement device 1 in such a way that it cannot unintentionally exit out of the tip of the sheath 3, but after the lock is released, the placement device 1 can be driven out at a selectable length.

It will be apparent to those skilled in the art that numerous modifications and variations of the foregoing versions of the invention are possible in light of the above discussion. The disclosed versions are presented for purposes of illustration only. Therefore, it is the intent to cover all such modifications and alternate versions as may be literally or equivalently encompassed by the claims below.

What is claimed is:

1. A system for placement of a medical implant in a living body, the system including a medical implant having:
a. an elongated implant housing extending between a distal implant end and a proximal implant end;
b. one or more electrical poles at the distal implant end for emitting electrical pulses into tissue of the living body; and
c. an implant passage extending between openings at the distal and proximal implant ends.

2. The system of claim 1 wherein the distal implant end includes a screw thereon, the screw being configured to anchor within tissue of the living body.

3. The system of claim 2 wherein the screw defines at least one of the electrical poles.

4. The system of claim 1 wherein the medical implant further includes a seal within the implant passage at or adjacent to the distal implant end, the seal being penetrable by an anchor to closely fit about the anchor penetrating the seal.

5. The system of claim 1 wherein the medical implant further includes a stop situated within the implant passage adjacent to the seal, wherein:
a. the implant passage narrows between the stop and the distal implant end, and
b. the stop includes a thrust bearing.

6. The system of claim 1 further including a placement device having:
a. an elongated device body extending between a distal device end and a proximal device end, the device body being configured to pass through an aditus into the living body with the proximal device end remaining outside of the living body,
b. an anchor on the distal device end, the anchor being configured to anchor within tissue of the living body in a removable manner.

7. The system of claim 6 wherein the anchor has a pointed tip.

8. The system of claim 6 wherein the anchor has a bend therein with a radius of 5 mm to 20 mm.

9. The system of claim 6 wherein the anchor has a bend therein, the bend curving arcuately to at least substantially conform to an arc of a circle with an angle measuring is between 70° to 90°.

10. The system of claim 6 wherein the device body and anchor are formed of a flexible wire.

11. The system of claim 10 wherein the wire has a diameter less than or equal to 0.4 mm.

12. The system of claim 6 further including a sheath having:
a. an elongated sheath housing extending between a proximal sheath end and a distal sheath end, the sheath housing being sized to at least partially fit within the implant passage; and
b. a primary sheath passage extending between openings at the distal and proximal sheath ends, the primary sheath passage being configured to translatably receive the device body therein.

13. The system of claim 12 further including a seal within the implant passage at or adjacent to the distal sheath end, the seal being penetrable by the anchor of the device body with the seal closely fitting about the anchor.

14. The system of claim 12 further including a locking unit at the proximal sheath end, the locking unit being selectively engageable to the device body to prevent displacement between the sheath and the device body.

15. The system of claim 6 further including a sheath having:
a. an elongated sheath housing extending between a proximal sheath end and a distal sheath end;
b. a primary sheath passage extending between primary openings at the distal and proximal sheath ends, the primary sheath passage being configured to receive the medical implant therein;
c. a secondary sheath passage extending between secondary openings at the distal and proximal sheath ends, the secondary sheath passage being configured to receive the placement device therein.

16. The system of claim 15 further including a locking unit at the proximal sheath end, the locking unit being selectively engageable to one or more of:
a. the implant housing of the medical implant within the primary passage, and
b. the device body of the placement device within the secondary sheath passage.

17. The system of claim 1 further including a sheath having:
a. an elongated sheath housing extending between a proximal sheath end and a distal sheath end, the sheath housing being sized to at least partially fit within the implant passage; and
b. a primary sheath passage extending between primary openings at the distal and proximal sheath ends, with the primary sheath passage opening onto the implant passage.

18. A system for placement of a medical implant in a living body, the system including a placement device having:
a. an elongated device body extending between a distal device end and a proximal device end, the device body being configured to pass through an aditus into the living body with the proximal device end remaining outside of the living body, and
b. an anchor on the distal device end, the anchor being configured to anchor within tissue of the living body in a removable manner.

19. The system of claim 18 wherein the anchor is bent to terminate in a pointed tip.

20. The system of claim 18 further including a sheath having an elongated sheath housing with a distal sheath end, a proximal sheath end, and a sheath passage extending between openings at the distal and proximal sheath ends, the sheath passage being configured to translatably receive the device body therein.

21. The system of claim 20 further including a seal at or adjacent to the distal sheath end, the seal being penetrable by the anchor of the device body with the seal closely fitting about the anchor.

22. The system of claim 20 further including a locking unit at the proximal sheath end, the locking unit being selectively engageable to the device body to prevent displacement between the sheath and the device body.

23. The system of claim 20 further including a medical implant, the medical implant having an elongated implant housing extending between a distal implant end and a proximal implant end, and including:
a. an implant passage extending between openings at the distal and proximal implant ends, with the implant passage being in communication with the sheath passage; and
b. one or more electrical poles at the distal implant end for emitting electrical pulses into tissue of the living body.

24. The system of claim 23 wherein the distal implant end further includes a screw thereon, the screw being configured to anchor within tissue of the living body.

25. The system of claim 24 wherein the screw defines at least one of the electrical poles.

26. The system of claim 20 further including a medical implant, the medical implant having an elongated implant housing extending between a distal implant end and a proximal implant end, and including:
a. an implant passage extending between openings at the distal and proximal implant ends, with the implant passage being in communication with the sheath passage, and
b. a stop situated within the implant passage adjacent to the distal implant end, wherein:
(1) the implant passage narrows between the stop and the distal implant end, and
(2) the stop includes a thrust bearing.

27. The system of claim 26 further including a seal situated within the implant passage between the stop and the distal implant end, the seal being penetrable by the anchor of the device body with the seal closely fitting about the anchor.

28. The system of claim 18 further including a medical implant, the medical implant having an elongated implant housing extending between a distal implant end and a proximal implant end, and including:
a. an implant passage extending between openings at the distal and proximal implant ends, the implant passage being configured to translatably receive the device body therein, and
b. a seal at or adjacent to the distal implant end, the seal being penetrable by the anchor of the device body with the seal closely fitting about the anchor.
